(19) United States
(12) Patent Application Publication
(10) Pub. No.: US 2012/0141576 A1
(43) Pub. Date: Jun. 7, 2012

(54) Treatment of Dermatologic Skin Disorders

(76) Inventor: Benjamin Johnson, Evergreen, CO (US)

(21) Appl. No.: 11/686,883

(22) Filed: Mar. 15, 2007

(51) Int. Cl. A61K 9/127; A61K 31/203; A61P 17/06; A61P 17/12; A61P 17/10; A61K 31/07; A61P 17/00

(52) U.S. Cl. 424/450; 514/725; 514/557

(57) ABSTRACT

Retinal and its derivatives are incorporated with liposomes in therapeutic compositions for topical application to prevent or alleviate the conditions and symptoms of cosmetic and dermatologic disorders are described as follows. In particular, retinal and its derivatives are combined with liposomes to increase the penetration of the retinal into the skin, to provide hydration of the skin during treatment, to reduce irritation of the skin during the application of the retinal, for use as a chemical skin peel and other embodiments and uses. The present invention includes utilizing retinol products, and particularly retinal in combination with liposomes each of these embodiments and uses separately as well as in combinations with one another.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. These disorders range from age spots, wrinkles, warts, acne, eczema, keratoses, psoriasis, xeorsis, aging skin, biochemical disorders within the skin and many other disorders.

These skin care products use a multitude of ingredients arranged in numerous formulations. These products have had varying amounts of successes in treating dermatologic and cosmetic skin disorders.

One area of products that have shown promise is the use of retinoidal compounds. Retinoidal compounds such as retinal, retinol and retinoic acid, have been used in therapeutic compositions for topical application to prevent or alleviate the conditions and symptoms of cosmetic and dermatologic disorders. Retinol is presently the preferred derivative since it endogenous compound naturally occurring in the human body and has a greater safety margin than other retinoids

However, retinoidal compounds tend to be very irritating which discourage their continued use for treatment of skin compounds. Lower doses are often used in order to increase compliance with the use of these compounds. However this decreases the efficacy of these compounds. Attempts have been made to alleviate these side effects by using certain derivates of retinoic acid and by the use of different types of delivery systems.

One such type of delivery system is the use of liposomes to contain retinoic acid, retinol, retinaldehhyde and other related retinoid compounds. These previously used liposomes may be phospholipids, single chain nonphospholipids or zwitterionic surfactants. The use of liposome encased retinoid compounds have been found to reduce the skin irritation that occurred with the use of retinoid compounds used alone. The preparation of liposomes, with entrapped solutes, was demonstrated for the first time in 1965 in a published paper (J. Mol. Biol. 13:238-252, 1965) by Prof. A. D. Bangham of the United Kingdom.

Skin irritation is not the only problem that exist in the previous skin care products. Other problems arise in the degradation of the retinoidal compounds. This may lead to serious side effects to the skin. Retinol is structurally unstable and is easily metabolized with exposure to light, air, heat, metal ions and other environmental factors. Oxidation of retinol metabolizes the retinol to retinoic acid which may cause toxicity to the skin. Thus the stability of the retinoid compounds is critical to its use.

Another problem is the penetration of the retinols into the deeper skin levels. Few topically applied skin care products are capable of penetrating the skin in therapeutically effective concentrations. Retinols and specifically retinal are known to be poorly penetrated into the skin. Typical skin care products containing retinols have only two percent penetration into the skin. This means that ninety eight percent remains in the superficial layers which results in drying of the skin and over-exfoliation. Enhancing the amounts of the retinoid compounds to increase the penetration transdermally results in unwanted irritation. Attempts have been made to add penetration enhancing solvents such as glycerol but have not been substantially successful. Solvents such as glycerol irritate the skin and create discomfort to the user.

Skin care products that utilize retinols also dehydrate the skin. This not also causes dry skin but leads to skin irritation. Retinols are also known to increase photosensitivy which further increases skin dehydration. Many skin care products add moisturizers to combat skin dehydration but these also introduce oils and contaminants into the skin pores as well as reduce penetration into the skin.

Chemical peels are also popular for therapeutically treating a wide spectrum of skin disorders. These chemical peels apply corrosive chemicals to the surface of the epidermis layer of the skin to remove epidermal and dermal skin cells to treat these skin disorders. Typical chemical peels use alpha-hydroxy acids, salicylic acid, lactic acids and other such products. Problems with chemical peels is that the skin does suffer damage during the process such as discoloration and skin irritation that remains for sometime.

Thus a problem exists in providing topical skin care products containing retinols and particularly retinal that have increased efficacy in the treatment of many dermatologic and cosmetic skin disorders.

### SUMMARY OF THE INVENTION

The present invention solves these and other problems by providing retinal, retinal derivatives as well as any other retinoids with liposomes. The present invention may in various embodiments be used to increase the efficacy of the use of retinal for therapeutically and cosmetically treating many skin disorders.

A preferred embodiment of the present invention increases the efficacy of a topical skin care product by increasing the penetration of retinal or its derivatives into the skin. Compositions containing retinal or its derivatives are coated or mixed with liposomal materials. The liposomal retinal compound has been shown to increase the penetration of the retinal or retinal derivatives by ten fold which not only increases the efficacy of the retinal product but also reduces the amount of retinal in the superficial skin layers. This greatly reduces the irritation in these layers as well as increasing the moisture in the skin.

Another preferred embodiment of the present invention reduces the dehydration of the skin from using retinal or its derivatives. Compositions containing retinal or its derivatives are coated or mixed with liposomal materials. Liposomes have a natural affinity for water which assists in increasing the moisture in the skin during topical application of the liposomal retinal composition. Also, as discussed above, the liposomal retinal composition has increased penetration which reduces the retinal in the superficial skin layer which in turn decreases the dehydration of the skin.

Another preferred embodiment of the present invention reduces the irritation to the skin in using retinal or retinal derivatives. Compositions containing retinal or its derivatives are coated or mixed with liposomal materials. The reduction of retinal in the superficial skin layers by the deeper penetration of retinal using the liposomal formulation also decreases the irritation of the skin. Also the additional hydration of the skin using the liposomal formulation reduces the irritation as well.

Another preferred embodiment of the present invention utilizes liposomal retinal compositions as a chemical peel. Compositions containing retinal or its derivatives are coated or mixed with liposomal materials. The composition is used as strong stimulant of the dermis as well as promoting temporary exfoliation to remove areas of skin layers that may be damaged. The increased penetration of the liposomal retinal composition increases the efficacy of the chemical peel while also reducing the dehydration and irritation of the process. This also allows this chemical peel to affect the skin without using harsh acids which is the primary cause of unwanted side effects like post-inflammatory hyperpigmentation and scarring.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

It has now been discovered that retinal and its derivatives can be therapeutically useful on topical administration against varieties of cosmetic and dermatologic conditions and disorders including oily skin, age spots, wrinkles, warts, acne, eczema, seborrheic keratoses, psoriasis, dandruff, xerosis, inflammatory and pruritic skin, disturbed keratinization, skin changes associated with aging and viral infections.

In accordance with the present invention, retinal and its derivatives are incorporated with liposomes in therapeutic compositions for topical application to prevent or alleviate the conditions and symptoms of cosmetic and dermatologic disorders are described as follows. In particular, retinal and its derivatives are combined with liposomes to increase the penetration of the retinal into the skin, to provide hydration of the skin during treatment, to reduce irritation of the skin during the application of the retinal, for use as a chemical skin peel and other embodiments and uses. The present invention includes utilizing retinol products, and particularly retinal in combination with liposomes each of these embodiments and uses separately as well as in combinations with one another.

Retinal and Retinal Derivatives

The present invention utilizes retinol products and in particular retinal and retinal derivatives such as those described in U.S. Pat. Nos. 5,492,935 and 5,093,360 which are incorporated herein by reference and further described below. The present invention is described below in descriptive embodiments utilizing retinal and retinal derivatives. However, it is to be expressly understood that other forms of retinal and other retinol products are considered to be within the scope of the present invention as set forth in the claims.

Retinal which is also often called vitamin A aldehyde differs considerably from retinol and Retinoic acid. Retinol which is often called vitamin A alcohol and vitamin A is present in fish liver oils as an ester compound, retinyl palmitate which is used in many skin care products. Retinoic acid, also called vitamin A acid, is an oxidation product from retinol or retinal. Retinol and retinal are critical in the physiologic functions of vision, growth, reproduction and differentiation. Retinoic acid on the other hand does not contribute to vision and reproduction in humans and animals.

Retinal can exist in stereoisomeric forms, namely all-trans, 13-cis, 11-cis, 9-cis, 7-cis, 11,13-cis, 9,13-cis. However, the common form is all-trans retinal. Since retinal is chemically an aldehyde it can exist as hemiacetal and acetal forms by reacting with one or two molecules of an alcohol, such as methanol, ethanol or propanol. Such hemiacetal and acetal forms are usually more stable against alkali, and are more resistant to oxidation of the aldehyde group. Retinal may be shown by the following chemical structure: R.sub.1, R.sub.2 .dbd.alkyl, aralkyl, aryl or alkoxy group of saturated or unsaturated, straight or branched chain or cyclic form, having 1 to 25 carbon atoms, R.sub.3 .dbd.O or (OR.sub.4) (OR.sub.5), wherein R.sub.4, R.sub.5 .dbd.H, alkyl, aralkyl, aryl, diol or polyol group of saturated or unsaturated, straight or branched chain or cyclic form, having 1 to 25 carbon atoms; and the hydrogen atom attached to the carbon atom in the main chain as well as in R.sub.1, R.sub.2, R.sub.4 and R.sub.5 may be substituted by a halogen atom or a radical such as a lower alkyl, aralkyl, aryl or alkoxy having 1 to 9 carbon atoms. The hemiacetal and acetal forms are represented by —CH(OR.sub.4)(OR.sub.5) instead of —CHO. The compound may be called retinal hemiacetal when either R.sub.4 or R.sub.5 is H, and the compound is called retinal acetal when both R.sub.4 and R.sub.5 are for example alkyls or aralkyls. The compound is called retinal hydrate when both R.sub.4 and R.sub.5 are H.

Retinal and its derivatives may exist as stereoisomers such as all-trans, 13-cis, 11-cis, 9-cis, 7-cis, 11,13-cis and 9,13-cis forms.

The typical alkyl, aralkyl and aryl groups for R.sub.1 and R.sub.2 are for example, methyl, ethyl, propyl, isopropyl, butyl, pentyl, octyl, lauryl, stearyl, benzyl and phenyl etc. The halogen atoms are F, Cl, Br and I. The typical alkoxy groups are methoxy and ethoxy. The typical diol groups are glycol, propylene glycol and 1,3-butanediol. The typical polyol groups include glycerol, butanetriol, inositol and alditols; such as erythritol of tetraol, ribitol of pentaol, mannitol and sorbitol of hexaols.

Retinal or its derivative may react with an unsaturated chemical agent such as maleic anhydride, acetylene dicarboxylic acid or its ester, or hydroquinone to form a crystalline molecular complex called an adduct compound.

The representative retinal and its derivatives which may be useful for topical or systemic administration to improve cosmetic conditions or to alleviate dermatologic disorders are listed below:

All Trans Retinal; Retinal hydrate; Retinal methyl hemiacetal; Retinal ethyl hemiacetal; Retinal propyl hemiacetal; Retinal isopropyl hemiacetal; Retinal butyl hemiacetal; Retinal pentyl hemiacetal; Retinal octyl hemiacetal; Retinal benzyl hemiacetal; Retinal dimethyl acetal; Retinal diethyl acetal; Retinal dipropyl acetal; Retinal diisopropyl acetal; Retinal dibutyl acetal; Retinal dipentyl acetal; Retinal dioctyl acetal; Retinal dibenzyl acetal; Retinal hydroquinone adduct; Retinal maleic anhydride adduct; Retinal acetylene dicarboxylic acid ester adduct; Retinal propylene glycol hemiacetal and acetal; Retinal 1,2-o-isopropylidene glyceryl hemiacetal and acetal; Retinal 3-allyloxy-1, 2-propanediol hemiacetal and acetal; Retinal phytyl hemiacetal; Retinal diphytyl acetal; Retinal dodecyl hemiacetal; and Retinal didodecyl acetal.

Retinal and its derivatives may also be utilized in combination with or as additives to enhance therapeutic effects of other cosmetic or pharmaceutical agents to improve cosmetic conditions or alleviate the symptoms of dermatologic disorder. Cosmetic and pharmaceutical agents include those that improve or eradicate age spots, keratoses and wrinkles eradicating agents; antiacne agents; antibacterials; antiyeast agents; antifungal agents; antiviral agents; antidandruff agents; antidermatitis agents; antipruritic agents; antiinflammatory agents; antihyperkeratolytic agents; antidryskin agents; antipsoriatic agents; antiseborrheic agents; hair conditioners and hair treatment agents; antiaging and antiwrinkle agents; sunscreen agents; antihistamine agents; vitamins; corticosteroids, tanning agents; local anesthetics; hormones; retinoids and other dermatologicals.

Some examples of cosmetic and pharmaceutical agents are clotrimazole, miconazole, salicyclic acid, pramoxine, menthol, retinoic acid, hydrocortisone, hydrocortisone valerate, betamethasone valerate, betamethasone dipropionate, triamcinolone acetonide, fluocinonide, hydroquinone, clobetasol propionate, benzoyl peroxide, crotaminton, 5-fluorouracil, monobenzone, vitam A palmitate, vitamin E acetate and vitamin C.

Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

Accordingly, a preferred embodiment of the present invention provides cosmetic as well as medicinal compositions containing retinal or its derivatives coated in liposomal material which when topically or systemically administered will substantially improve and alleviate the symptoms of various cosmetic conditions or dermatologic disorders.

Another preferred embodiment of the present invention provides methods for treating various cosmetic conditions or dermatologic disorders with topical preparations containing retinal or its derivatives in conjunction with liposome material to improve penetration of the retinal into the skin, provide hydration during the application of the retinal or its derivatives, increase the efficacy of the retinal or its derivatives and/or to reduce oxidation and irritation normally seen with retinal in topical formulations.

Retinal and its derivatives of the instant invention may be formulated either for topical application or for systemic administration. In the topical preparations retinal and its derivatives may be formulated in aqueous or non-aqueous solution, gel, lotion, cream or ointment containing 0.0001 to 5 percent and preferably from 0.01 to 5 percent by weight of the total composition. When retinal and its derivatives are formulated in aqueous form, sodium sulfite, sodium bisulfite, sodium metabisulfite or other antioxidants may be added to stabilize retinal and its derivatives in aqueous compositions. Liposomal lecithin or a liposome substitute or other lipid preparations are added to the above solution with mixing until a uniform consistency is obtained. Butylated hydroxytoluene (BHT) or butylated hydroxyanisole (BHA) may be added to stabilize retinal and its derivatives in non-aqueous composition. To provide maximal stability of the therapeutic compositions antioxidants of both aqueous and nonaqueous types may also be incorporated into the compositions at the same time. For example, both sodium metabisulfite and BHT may be added to an aqueous acoholic solution containing retinal. The concentration of antioxidant may range from 0.01 to 5%. The most efficacious stabilizer is a liposomal coating which provides a lipid layer of protection and the concentration may range from 0.001 to 10%.

To prepare a typical aqueous solution, retinal or its derivative is dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, retinal or its derivative is dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing retinal or its derivative is prepared by first dissolving retinal or its derivative in ethanol, acetone, propylene glycol or other solvent. The solution thus prepared is then admixed with commonly available oil-in-water emulsions. BHT or sodium metabisulfite may be added to such emulsions to stabilize retinal or its derivative. Liposomes or liposome substitutes are added to this solution with mixing until a uniform consistency is achieved.

A typical gel composition is formulated by first dissolving retinal or its derivative in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of retinal for therapeutically and cosmetically treating many skin disorders.

A preferred embodiment of the present invention increases the efficacy of a topical skin care product by increasing the penetration of retinal or its derivatives into the skin. Compositions containing retinal or its derivatives are coated or mixed with liposomal materials as described above. The liposomal retinal compound has been shown to increase the penetration of the retinal or retinal derivatives by ten fold which not only increases the efficacy of the retinal product but also reduces the amount of retinal in the superficial skin layers. This greatly reduces the irritation in these layers as well as increasing the moisture in the skin.

Another preferred embodiment of the present invention reduces the dehydration of the skin from using retinal or its derivatives. Compositions containing retinal or its derivatives are coated or mixed with liposomal materials as described above. Liposomes have a natural affinity for water which assists in increasing the moisture in the skin during topical application of the liposomal retinal composition. Also, as discussed above, the liposomal retinal composition has increased penetration which reduces the retinal in the superficial skin layer which in turn decreases the dehydration of the skin.

Another preferred embodiment of the present invention reduces the irritation to the skin in using retinal or retinal derivatives. Compositions containing retinal or its derivatives are coated or mixed with liposomal materials as described above. The reduction of retinal in the superficial skin layers by the deeper penetration of retinal using the liposomal formulation also decreases the irritation of the skin. Also the additional hydration of the skin using the liposomal formulation reduces the irritation as well.

Another preferred embodiment of the present invention utilizes liposomal retinal compositions as a chemical peel. Compositions containing retinal or its derivatives are coated or mixed with liposomal materials as described above with retinal provided in the range of 0.5 to 5 percent by weight. The composition is used as chemical peel to remove areas of skin layers that may be damaged. The increased penetration of the liposomal retinal composition increases the efficacy of the chemical peel while also reducing the dehydration and irritation of the process.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

1-12. (canceled)

13. A method for minimizing the dehydration effect of retinal compounds from a topical skin care application, said method comprising the steps of:
providing a retinal compound in the skin care application; and
providing said retinal compound within a liposome compound to hydrate the skin during application of said retinal compound.

14. The method for minimizing the dehydration effect of retinal compounds from a topical skin care application of claim 13, wherein step of providing said retinal compound includes:
providing retinoidal compounds.

15. The method for minimizing the dehydration effect of retinal compounds from a topical skin care application of claim 13, wherein step of providing said retinal compound includes:
providing retinol.

16. The method for minimizing the dehydration effect of retinal compounds from a topical skin care application of claim 13, wherein step of providing said retinal compound includes:
providing retinoic acid.

17. The method for minimizing the dehydration effect of retinal compounds from a topical skin care application of claim 13, wherein step of providing said retinal compound includes:
providing derivatives of retinal.

18. The method for minimizing the dehydration effect of retinal compounds from a topical skin care application of claim 13, wherein step of providing said retinal compound includes:
providing said retinal compound in the range of between 0.001 percent to 5 percent by weight.

19. The method for minimizing the dehydration effect of retinal compounds from a topical skin care application of claim 13, wherein step of providing said retinal compound includes:
providing said retinal compound in the range of between 0.01 percent to 5 percent by weight.

20. The method for minimizing the dehydration effect of retinal compounds from a topical skin care application of claim 13, wherein step of providing said liposome compound includes:
providing lipid compounds having enhanced penetrating properties.

21. The method for minimizing the dehydration effect of retinal compounds from a topical skin care application of claim 13, wherein step of providing said liposome compound includes:
providing said liposome compound in the range of 0.1 percent to 20 percent by weight.

22. The method for minimizing the dehydration effect of retinal compounds from a topical skin care application of claim 13, wherein step of providing said liposome compound includes:
providing said liposome compound in the range of 2 percent to 5 percent by weight.

23. The method for minimizing the dehydration effect of retinal compounds from a topical skin care application of claim 13, wherein step of providing said liposome compound includes:
providing phosphatidycholine.

24. The method for minimizing the dehydration effect of retinal compounds from a topical skin care application of claim 13, wherein step of providing said liposome compound includes:
providing a lipid compound having a high affinity for water.

25. A method of increasing the efficacy of a skin care application, said method comprising the steps of:
providing a retinal compound in the skin care application; and
increasing the transdermal penetration of said retinal compound and moisturizing the skin during said transdermal penetration by providing said retinal compound within a liposome compound to increase the transdermal penetration of said retinal compound without increasing the irritation of the skin.

26. The method of increasing the efficacy of a topical skin care application of claim 25, wherein step of providing said retinal compound includes:
providing retinoidal compounds.

27. The method of increasing the efficacy of a topical skin care application of claim 25, wherein step of providing said retinal compound includes:
providing retinol.

28. T The method of increasing the efficacy of a topical skin care application of claim 25, wherein step of providing said retinal compound includes:
providing retinoic acid.

29. The method of increasing the efficacy of a topical skin care application of claim 25, wherein step of providing said retinal compound includes:
providing derivatives of retinal.

30. The method of increasing the efficacy of a topical skin care application of claim 25, wherein step of providing said retinal compound includes:
providing said retinal compound in the range of between 0.001 percent to 5 percent by weight.

31. The method of increasing the efficacy of a topical skin care application of claim 25, wherein step of providing said retinal compound includes:
providing said retinal compound in the range of between 0.01 percent to 5 percent by weight.

32. The method of increasing the efficacy of a topical skin care application of claim 25, wherein step of providing said liposome compound includes:
providing lipid compounds having enhanced penetrating properties.

33. The method of increasing the efficacy of a topical skin care application of claim 25, wherein step of providing said liposome compound includes:
providing said liposome compound in the range of 0.1 percent to 20 percent by weight.

34. The method of increasing the efficacy of a topical skin care application of claim 25, wherein step of providing said liposome compound includes:
providing said liposome compound in the range of 2 percent to 5 percent.

35. The method of increasing the efficacy of a topical skin care application of claim 25, wherein step of providing said liposome compound includes:
providing phosphatidycholine.

36. The method of increasing the efficacy of a topical skin care application of claim 25, wherein step of providing said liposome compound includes:
providing a lipid compound having a high affinity for water.
